(19) United States
(12) Patent Application Publication
(10) Pub. No.: US 2025/0041380 A1
(43) Pub. Date: Feb. 6, 2025

(54) RECOMBINANT PROTEIN FOR SARS-COV2 DISEASE

(71) Applicant: Unichem Laboratories Limited, Mumbai (IN)

(72) Inventors: Dhananjay Sathe, Thane (IN); Saravanakumar Iyappan, Hyderabad (IN)

(21) Appl. No.: 18/718,862

(22) PCT Filed: Dec. 14, 2022
(86) PCT No.: PCT/IB2022/062211

§ 371 Date: Jun. 12, 2024

(30) Foreign Application Priority Data
Dec. 16, 2021 (IN) 202121058651

(51) Int. Cl. A61K 38/16; A61P 31/14

(52) U.S. Cl. CPC A61K 38/16; A61P 31/14

(57) ABSTRACT

The present invention relates to the protein for treatment of infectious disease caused by Coronaviridae. In particular, the invention relates to a recombinant Sclerotium rolfsii lectin for use in the treatment, reduction in progression and curing of SARS-COV2 disease in a subject in need thereof. Pharmaceutical compositions comprising said recombinant Sclerotium rolfsii lectin are also described. The recombinant Sclerotium rolfsii lectin protein may comprise an amino acid sequence having at least 70% homology to SEQ ID NO: 1.

### FIELD OF INVENTION

The present invention relates to the use of lectin proteins for the prevention, treatment and cure of SARS-Cov2 disease.

### BACKGROUND OF INVENTION

Severe acute respiratory syndrome virus 2 (SARS-COV-2) causes a respiratory disease that led to the fatal Coronavirus disease 2019 (COVID-19) pandemic. In late 2019, the SARS-COV-2 outbreak was first reported in Wuhan, China that later led to a true crisis worldwide (Huang et al., 2020). Coronaviruses (CoVs) are large enveloped non-segmented positive-sense RNA viruses. They generally cause mild enteric and respiratory diseases in animals and humans (Glass et al., 2004). Most human CoVs, such as hCoV-229E, OC43, NL63, and HKU1 usually cause only mild respiratory diseases (Fouchier et al., 2004). SARS-COV-2 causes acute, highly lethal pneumonia with clinical symptoms similar to those reported for SARS-COV and MERS-COV-2 (Fouchier et al., 2004). In contrast to SARS-COV, SARS-COV-2-infected patients rarely show prominent upper respiratory tract signs and symptoms. Based on current literature, most severe SARS-COV-2 cases progressed within 14-21 days after disease onset. Interestingly, these risk factors are similar to the reported risk factors (diabetes, hypertension, obesity) associated with MERS-COV related mortality, although MERS-COV respiratory disease occurred in younger individuals (Assiri et al., 2013; World Health Organization, 2013).

The coronavirus genome is comprised of ˜30000 nucleotides. It encodes four structural proteins, Nucleocapsid (N) protein, Membrane (M) protein, Spike(S) protein and Envelop (E) protein and several non-structural proteins (nsp) (FIG. 1). The capsid is the protein shell, inside the capsid, there is nuclear capsid or N-protein which is bound to the virus single positive strand RNA that allows the virus to hijack human cells and turn them into virus factories. To enter host cells, coronaviruses first bind to a cell surface receptor for viral attachment, subsequently enter endosomes, and eventually fuse viral and lysosomal membranes. A virus surface-anchored spike(S) protein attaches to angiotensin converting enzyme 2 (ACE2) receptors that is found on the surface of many human cells, including those in the lungs allowing virus entry. The coronavirus S protein is subjected to proteolytic cleavages by host proteases (i.e. trypsin and furin), in two sites located at the boundary between the S1 and S2 subunits (S1/S2 site). In a later stage happens the cleavage of the S2 domain (S2′ site) in order to release the fusion peptide. This event will trigger the activation of the membrane fusion mechanism. These clinical and epidemiological observations provide direction on the mechanism of disease and elucidate that the virus gains entrance into its host cell via the ACE2 receptor.

Viruses mutate, and SARS-COV-2 is no exception. Throughout the current global crisis, SARS-CoV-2 has been mutating at a rate of about 1-2 mutations per month. Various mutations of this virus are being reported across the world. The mutant form of the spike protein can affect the viral infectivity. Mutations in the Spike protein have been reported to play a role in the antigenicity of the virus and these mutations have different effects on different cell lines (Qianqian Li et. al, 2020). Virus mutation could prolong the pandemic and raise the risk of further mutations of the virus emerging, possibly undermining the efficacy of existing vaccines in individuals over time; and the rise of new variants pose serious concern over mutations with high vaccine escape potential.

The spectrum of medical therapies to Combat coronavirus disease 2019 (COVID-19) is growing and evolving rapidly. The range of treatment options currently available for Covid 19 includes Convalescent plasmatherapy, corticosteroid (steroid) medication (Dexomethasone), Azithromycin, Hydroxychloroquine and chloroquine, Ivermectin Kaletra Monoclonal antibodies (MABs) Bamlanivimab (LY-CoV555) and combination of Bamlanivimab and etesevimab: Casirivimab and imdevimab, Tocilizumab, IL-6 inhibitors, kinase inhibitors such as Acalabrutinib (Olumiant) Ruxolitinib (Jakafi) Tofacitinib (Xeljanz); Repurposed medications oseltamivir favipiravir and vaccines such as Covishield, Covaxin, moderna vaccine, Sputnik, sinovac, Janssen COVID-19 etc. Though many treatment options available, the cost of the treatments and rapid mutations of the COVID 19 bottlenecks the current treatment options for COVID 19.

The presence of glycoproteins on the viral envelope opens a wide range of possibilities for the application of lectins to address some urgent problems involved in this pandemic (LuísCláudio et al. 2021). The envelope of SARS-COV-2 exhibits glycoproteins such as spike (Sprotein) and membrane (M-protein) proteins (Ahmed, S. F., Quadeer, A. A., et al. 2020, Shajahan, A., Supekar, N. T., et al. 2020) (Supplementary FIG. 1A). These structuralproteins (specially S-protein) exert central roles in viral pathogenesis and are pointed as important targets for neutralizing antibodies, vaccine and drug design (Ahmed, S. F., Quadeer, A. A., et al. 2020, Bagdonaite, I. and Wandall, H. H. 2018). A study evaluated the in vitro antiviral activity of 33 plant lectins towards coronaviruses (SARS-COV and feline infectious peritonitis virus). Mannose-binding agglutinins showed the highest anti-SARS-COV effects. Other plant lectins have been shown to exhibit inhibitory action towards other coronaviruses. Examples include the following mannose-binding lectins: Concanavalin A (Con A), Hippeastrum hybrid agglutinin (HHA), Galanthus nivalis agglutinin (GNA or GNL) (Greig, A. S. and Bouillant, A. M. 1977, Hsieh, L. E., Lin, C. N., et al. 2010, Nguyen, T. D., Bottreau, E., et al. 1987, van der Meer, F. J., de Haan, C. A., et al. 2007a). One of these studies highlighted the importance of glycosylation in the sensibility of two types of coronaviruses (mouse hepatitis virus and feline infectious peritonitis virus) towards GNA, UDA and HHA. In this case, the inhibition of host mannosidases resulted in the improved antiviral activity of the mannose- and GlcNAc-binding lectins (van der Meer, F. J., de Haan, C. A., et al. 2007b).

Non-plant derived agglutinins are also pointed as promising agents against coronaviruses, e.g. the mannose-binding-lectins cyanovirin-N (from cyanobacterium Nostocellipsosporum) and griffithsin (from red marine alga Griffithsia sp.) (O'Keefe, B. R., Giomarelli, B., et al. 2010, van der Meer, F. J., de Haan, C. A., et al. 2007a). However, only griffithsin (GRFT) has been evaluated against SARS-COV and MERS-COV. This protein binds to multiple sites of SARS-CoV and MERS-Cov glycoproteins with high affinity and inhibits viral entry (Millet, J. K., Seron, K., et al. 2016, O'Keefe, B. R., Giomarelli, B., et al. 2010). Additionally, this lectin also reduced the mortality and the severity of lethal pulmonary infection induced by SARS-COV in mice. This effect is associated with the decrease of proinflammatory cytokines in infected lung tissue (O'Keefe, B. R., Giomarelli, B., et al. 2010). Also some of the mannose binding lectin, plant lectin such as phytohemagglutininsucc-Con A, lentil lectin and lectin isolated from Lablab purpureus (FRIL) for the treatment or prevention of SARS-Cov 2 has been studied in the patent application WO2021/222965, CN113262294, CN113274484 WO2021/226294. Despite the potential antiviral activity of the aforementioned lectins, some limitations should be considered regarding their therapeutic utility. One practical issue is the rapid degradation of these macromolecules, imposing the need for multiple doses to maintain a therapeutic level.

Sclerotium rolfsii lectin (SRL) is a lectin that has been isolated from the sclerotial bodies of the soil-borne phytopathogenic fungus S. rolfsii. SRL has specificity towards Thomsen-Friedenreich (TF) antigen, Tn antigen and its extended core structures. TF antigen is a disaccharide (Galβ1→3GalNAc-α-Ser/Thr) that is over expressed on the cell surface of various human cancer cells. Tn antigen is a monosaccharide (GalNAc-α-Ser/Thr). TF or T antigen is an intermediate structure in the biosynthesis of complex O-linked oligosaccharides. Its formation is carried out by the addition of galactose (Gal) from UDP-Gal to the precursor structure GalNAc (Tn antigen). Applicant's earlier application WO 2010/095143 discloses recombinant lectin variants Rec-2 and Rec-3, which are derived from the native SRL sequence by the substitution of 3 or 5 amino acids respectively. The crystal structure of these variants has been reported (Peppa et al., Molecules. 2015 Jun. 12; 20 (6): 10848-65). WO 2014/203261 discloses a recombinant lectin variant derived from the native SRL sequence by the substitution of 12 amino acids. Also the anti-inflammatory property of the recombinant Sclerotium rolfsii lectin has also been disclosed in the applicant earlier application WO/2020/074984.

Today there are very few approved biologics for the treatment of SARS-COV2 disease. They are highly costly and are scarcely available due to less production and high demand. Thus there is an unmet need to provide an option for the treatment of SARS-Cov2, wherein the option is highly specific towards its action, is cheap and effectively accomplishes the market needs.

### OBJECT OF INVENTION

Therefore an object of the present invention is to provide a recombinant Sclerotium rolfsii lectin for use in the treatment or prevention of SARS-COV2 disease in a subject in need thereof.

Therefore an object of the present invention is to provide a method of treatment using a recombinant Sclerotium rolfsii lectin for treatment or prevention of SARS-COV2 disease in a subject in need thereof.

Therefore an object of the present invention is to provide a composition comprising a recombinant Sclerotium rolfsii lectin for treatment or prevention of SARS-COV2 disease in a subject in need thereof.

### SUMMARY OF INVENTION

According to a first aspect of the present invention, there is provided are combinant Sclerotium rolfsii lectin for use in the treatment or prevention of aninfectious disease caused by Coronaviridae in a subject in need thereof.

According to a second aspect of the invention, there is provided a pharmaceutical composition comprising a recombinant Sclerotium rolfsii lectin and a pharmaceutically acceptable diluent or excipient. Optionally, the pharmaceutical composition may comprise a further therapeutic agent.

According to a third aspect of the invention, there is provided a method of treatment or prevention of infectious disease caused by Coronaviridae in a subject, the method comprising administeringa recombinant Sclerotium rolfsii lectin, or the pharmaceutical composition of the second aspect of the invention, to the subject.

According to a fourth aspect of the invention, there is provided a use of a recombinant Sclerotium rolfsii lectin or pharmaceutical composition comprising a recombinant Sclerotium rolfsii lectin for the treatment or prevention of infectious disease caused by Coronaviridae in a subject.

According to yet another aspect of the present invention, there is provided a recombinant Sclerotium rolfsii lectin protein for use in the treatment or prevention of infectious disease caused by virus selected from SARS-COV2, SARS-COV, MERS-COV, HCOV-229E, HCoV-OC43, HCoV-NL63, HCoV-HKU1, and mutants thereof.

According to yet another aspect of the present invention, there is provided a recombinant Sclerotium rolfsii lectin protein for use in the treatment or prevention of infectious disease caused by virus is selected from SARS-COV2 and mutants thereof.

The lectin may comprise an amino acid sequence having at least 60% homology to SEQ ID NO: 1. In some embodiments the lectin comprises an amino acid sequence having at least 70%, 80%, 90%, 95%, 96%, 97%, 98% or 99% homology to SEQ ID NO: 1. In some embodiments, the lectin comprises the amino acid sequence of SEQ ID NO: 1. The lectin protein may be recombinant.

According to yet another aspect of the present invention, there is provided a recombinant Sclerotium rolfsii lectin protein which comprises an amino acid sequence selected from the group consisting of SEQ ID NO. 2, SEQ ID NO. 3 or SEQ ID NO. 4.

According to yet another aspect of the present invention, there is provided a recombinant Sclerotium rolfsii lectin protein for use in treatment or prevention of SARS-Cov2 and its mutants or variants thereof, wherein the recombinant lectin protein is capable of inhibiting the viral entry by blocking SpikeS1 protein of SARS-COV2 with ACE2 and inhibiting or modulating the cytokine storm induced by mutant SpikeS1 proteins (SARS-COV-2 derived) induced cytokine storm in lung and immune cells. The recombinant Sclerotium rolfsii lectin protein interrupts SARS-COV-2 entry in cell via AT1R downregulation and significantly inhibits relevant proteases in pathogenesis of Covid and also the recombinant Sclerotium rolfsiiprotein significantly inhibits cytokines such as IL6 and IL8 as compared to control, in lung cells (A549) and immune cells (THP-1).

Another aspect of the present invention is to provide recombinant Sclerotium rolfsii lectin protein for the treatment of SARS-COV2.

Yet another aspect of the present invention is to provide a method of treatment of SARS-COV2 and its mutant using recombinant Sclerotium rolfsii lectin protein.

Yet another aspect of the present invention is to provide a method to cure or reduce the effect of SARS-COV2 in subject suffering from the diseases. The present invention also relates to a method of treatment of SARS-COV2. The method comprises administration of a lectin protein. The lectin protein may be administered in an amount in the range of about 0.001 mg to 50 mg/kg body weight of the subject. The lectin protein may be admininstered as such or in a pharmaceutically acceptable form.

A lectin protein may be administered to the subject by injecting the solution or suspension intravenously, intramuscularly, intraperitoneally, subcutaneously, intradermally, by depot injection, intrathecally, transdermally, sublingually or by oral, topical or inhalation methods.

The term “lectin” as used herein refers to a carbohydrate-binding protein.

The term “protein” as used herein refers to a polymer of amino acid residues.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code and include the proteinogenic amino acids. Naturally occurring amino acids also include those modified after translation in cells. Synthetic amino acids include non-canonical amino acids such as selenocysteine and pyrrolysine. Typically synthetic amino acids are not proteinogenic amino acids.

It is understood that amino acids can be grouped according to different biochemical properties. Examples include: the polar amino acids, the non-polar amino acids, the acidic amino acids and the basic amino acids. In one embodiment, the amino acid used for the amino acid modification is at least one selected from the group consisting of, but not limited to: polar, non-polar, acidic, basic, selenocysteine, pyrrolysine and non-canonical.

The terms “homology” or “homologous” as used herein refer to two or more referenced entities that share at least partial identity over a given region or portion. Areas, regions or domains of homology or identity refer to a portion of two or more referenced entities that share homology or are the same. Thus, where two sequences are identical over one or more sequence regions they share identity in these regions. Substantial homology refers to a molecule that is structurally or functionally conserved such that it has or is predicted to have at least partial structure or function of one or more of the structures or functions (e.g., a biological function or activity) of the reference molecule, or a relevant/corresponding region or portion of the reference molecule to which it shares homology.

In one embodiment, the percentage “homology” between two sequences is determined using the BLASTP algorithm with default parameters (Altschul et al. Nucleic Acids Res. 1997 Sep. 1; 25 (17): 3389-402). In particular, the BLAST algorithm can be accessed on the internet using the URL: https://blast.ncbi.nlm.nih.gov/Blast.cgi. In an alternative embodiment, for global sequence alignments, percentage homology between two sequences is determined using the EMBOSS Needle algorithm using default parameters. In particular, the EMBOSS Needle algorithm can be accessed on the internet URL: using the https://www.ebi.ac.uk/Tools/psa/emboss_needle/.

Unless otherwise indicated, the term “homology” is used interchangeably with the term “sequence identity” in the present specification.

The term “Coronaviridae” refers to a large, enveloped, single-stranded RNA viruses. They are the largest known RNA viruses, with genomes ranging from 25 to 32 kb and virions of 118-140 nm in diameter.

The term spike protein or “S” protein used interchangeably is a large type I transmembrane protein ranging from 1,160 up to 1,400 amino acids. In addition, this protein is highly glycosylated as it contains 21 to 35 N-glycosylation sites. Spike proteins assemble into trimers on the virion surface to form the distinctive “corona”, or crown-like appearance. The ectodomain of all CoV spike proteins share the same organization in two domains: a N-terminal domain named S1 that is responsible for receptor binding and a C-terminal S2 domain responsible for fusion. CoV diversity is reflected in the variable spike proteins (S proteins), which have evolved into forms differing in their receptor interactions and their response to various environmental triggers of virus-cell membrane fusion. The spike protein or “S” protein is derived from SARS-CoV2 or its mutants.

The term “ACE2” refers to a protein on the surface of many cell types. It is an enzyme that generates small proteins—by cutting up the larger protein angiotensinogen—that then go on to regulate functions in the cell. ACE2 is present in epithelium in the nose, mouth and lungs. In the lungs, ACE2 is highly abundant on type 2 pneumocytes, an important cell type present in chambers within the lung called alveoli, where oxygen is absorbed and waste carbon dioxide is released. ACE2 helps modulate the many activities of a protein called angiotensin II (ANG II) that increases blood pressure and inflammation, increasing damage to blood vessel linings and various types of tissue injury. ACE2 converts ANG II to other molecules that counteract the effects of ANG II.

The terms “administer,” “administering,” or “administration,” as used herein refers to implanting, absorbing, ingesting, injecting, inhaling, or otherwise introducing an inventive compound, or a pharmaceutical composition thereof.

An “effective amount” of a compound or any active ingredient as described herein of refers to an amount sufficient to elicit a desired biological response, i.e., treating the condition. As will be appreciated by those of ordinary skill in this art, the effective amount of a compound may vary depending on such factors as the desired biological endpoint, the pharmacokinetics of the compound, the condition being treated, the mode of administration, and the age and health of the subject. An effective amount encompasses therapeutic and prophylactic treatment.

The composition described herein can be mixed with a pharmaceutically acceptable carrier or excipient to form a pharmaceutical composition for use in inhibiting a coronavirus and/or treating infection caused by a SARS-COV2 virus.

The term “inhibiting,” “inhibition,” “inhibit,” “inhibitor,” and the like, refer to the ability of an anti-coronavirus agent to reduce, slow, halt, or prevent activity of a particular biological process (e.g., coronavirus replication) in a cell relative to a control vehicle. In some instances, an anti-coronavirus agent can inhibit the level of viral replication by at least 20% (e.g., 30%, 40%, 50%, 60%, 70%, 80%, 90%, or 95%).

The term “treating” or “treatment” refers to administering one or more anti-coronavirus agent (e.g., the composition described herein) to a subject (e.g., a human patient), who has SARS-Cov2 infection, a symptom of or a predisposition toward it, with the purpose to confer a therapeutic effect, e.g., to cure, relieve, alter, affect, ameliorate, or prevent the infection, the symptom of or the predisposition toward it. Such a subject can be identified by a health care professional based on results from any suitable diagnostic method.

The term “infectious disease” is the invasion of an organism's body tissues by disease-causing agents, their multiplication, and the reaction of host tissues to the infectious agents and the toxins they produce. An infectious disease, also known as a transmissible disease or communicable disease, is an illness resulting from an infection. Infections are caused by infectious agents that includes Bacteria, Fungi, Viral, Prions, Parasites and Arthropods.

The term “SARS-Cov2” or “Covid-19” or “Covid” or “corona virus disease” interchangeably used, refers to the infectious disease caused by the virus SARS-COV2 or its mutants.

The term “mutant” is defined as an organism or a new genetic character arising or resulting from an instance of mutation, which is generally an alteration of the DNA sequence of the genome or chromosome of an organism. It is a characteristic that would not be observed naturally in a specimen. The term mutant is also applied to a virus with an alteration in its nucleotide sequence whose genome is in the nuclear genome.

### BRIEF DESCRIPTION OF THE ACCOMPANYING SEQUENCES

SEQ ID NO. 1: represents the native S. rolfsii
lectin amino acid sequence.
TYKITVRVYQTNPNAFFHPVEKTVWKYANGGTWTITDDQHVLTMGGSGTS
GTLRFHADNGESFTATFGVHNYKRWCDIVTNLAADETGMVINQQYYSQKN
REEARERQLSNYEVKNAKGRNFEIVYTEAEGNDLHANLIIG
SEQ ID NO: 2: represents a variant of the S.
rolfsii lectin amino acid sequence (reported as
Rec-2 in WO 2010/095143).
TYKITVRVYQTNPDAFFHPVEKTVWKYANGGTWTITDDQHVLTMGGSGTS
GTLRFHADNGESFTATFGVHNYKRWCDIVTNLAADETGMVINQQYYSQKN
REEARERQLSNYQVKNAKGRNFQIVYTEAEGNDLHANLIIG
SEQ ID NO: 3: represents a variant of the S.
rolfsii lectin amino acid sequence (reported as
Rec-3 in WO 2010/095143).
VYKITVRVYQTNPDAFFHPVEKTVWKYANGGTWSITDDQHVLTMGGSGTS
GTLRFHADNGESFTATFGVHNYKRWCDIVTNLAADETGMVINQQYYSQKN
REEARERQLSNYQVKNAKGRNFQIVYTEAEGNDLHANLIIG
SEQ ID NO: 4: represents a variant of the S.
rolfsii lectin amino acid sequence (reported in
WO 2014/203261).
VYKITVRVYQTNPDAFFHPVEKTVWKYADGGTWSITDDQHVLTMGGSGTS
GTLRFHADNGESFTATFGVHDYKRWCDIVTDLAADETGMVINQEYYSEKD
REEARERQNSNYEVKDAKGRNFEIVYTEAEGNDLHADLIIG

### LIST OF TABLES

Table 1. Effect of recombinant Sclerotium rolfsil lectin of SEQ ID NO: 2 on inhibition of binding of SpikeS1 protein with ACE2 protein
Table 2. Effect of recombinant Sclerotium rolfsu lectin of SEQ ID NO: 2 on viability of Immme (THP-1) after 48 h
Table 3. Effect of recombinant Sclerotium rolfsii lectin of SEQ ID NO: 2 on viability of Lung (A549) cells after 48 h
Table 4: Inhibitory effect of Recombinant Sclerotium rolfsii lectin of SEQ ID NO: 2 for Injection on levels of cytokines induced by mutant SARS-COV-2-spike S1 (E484K) protein in Immune cells (THP-1)
Table 5: Inhibitory effect of Recombinant Sclerotium rolfsii lectin of SEQ ID NO: 2 for Injection on levels of cytokines induced by mutant SARS-COV-2-spike S1 (N501Y) protein in Lung cells (A549)
Table 6: Effect of recombinant Sclerotium rolfsii lectin of SEQ ID NO: 2 for Injection on inhibition of Cathepsin L
Table 7: Effect of recombinant Sclerotium rolfsii lectin of SEQ ID NO: 2 for Injection on inhibition of 3CL
Table 8: Effect of recombinant Sclerotium rolfsii lectin of SEQ ID NO: 2 for Injection on inhibition of DPP4
Table 9: Inhibitory effect of Recombinant Sclerotium rolfsii lectin of SEQ ID NO: 2 on expression of inflammatory markers in Bladder cells (T24) induced by SpikeS1 protein (SARS-COV-2)
Table 10: Inhibitory effect of Recombinant Sclerotium rolfsii lectin of SEQ ID NO: 2 on expression of inflammatory markers in Cardiac cells (H9C2) induced by SpikeS1 protein (SARS-COV-2)
Table 11: Inhibitory effect of Recombinant Sclerotium rolfsii lectin of SEQ ID NO: 2 on expression of inflammatory markers in Colon cells (HT-29) induced by SpikeS1 protein (SARS-COV-2)
Table 12: Inhibitory effect of Recombinant Sclerotium rolfsii lectin of SEQ ID NO: 2 on expression of inflammatory markers in Endothelial cells (EA.hy926) induced by SpikeS1 protein (SARS-COV-2)
Table 13: Inhibitory effect of Recombinant Sclerotium rolfsii lectin of SEQ ID NO: 2 on expression of inflammatory markers in Kidney cells (HEK-293) induced by SpikeS1 protein (SARS-COV-2)
Table 14: Inhibitory effect of Recombinant Sclerotium rolfsii lectin of SEQ ID NO: 2 on expression of inflammatory markers in Bone cells (MG-63) induced by SpikeS1 protein (SARS-COV-2)
Table 15: Inhibitory effect of Recombinant Sclerotium rolfsii lectin of SEQ ID NO: 2 on expression of inflammatory markers in liver cells (HepG2) induced by SpikeS1 protein (SARS-COV-2)
Table 16: Inhibitory effect of Recombinant Sclerotium rolfsii lectin of SEQ ID NO: 2 on expression of inflammatory markers in pancreatic cells (PANC-1) induced by SpikeS1 protein (SARS-COV-2)

### DETAILED DESCRIPTION OF THE INVENTION

The present invention provides a recombinant Sclerotium rolfsii lectin for use in the treatment or prevention of infectious disease caused by Coronaviridaein a subject in need thereof.

The recombinant Sclerotium rolfsii lectin may comprise an amino acid sequence having at least 70% homology to SEQ ID NO: 1. In some embodiments, the amino acid sequence has at least 75%, 80%, 90%, 95%, 96%, 97%, 98% or 99% homology to SEQ ID NO: 1.

In some embodiments, the recombinant Sclerotium rolfsii lectin comprises an amino acid sequence selected from the group consisting of SEQ ID NO: 2, SEQ ID NO: 3 or SEQ ID NO: 4.

Methods for preparing recombinant proteins will be well-known to those skilled in the art. For example, a recombinant DNA molecule, such as a plasmid or viral vector, comprising a nucleic acid sequence encoding the lectin may be provided. The nucleic acid sequence may be operatively linked to a promoter which is capable of controlling expression of the lectin in a suitable host cell. The recombinant DNA molecule may be inserted into a suitable host cell using methods known in the art, for example by transformation. Suitable host cells include prokaryotic cells (e.g. E. coli) and lower eukaryotic cells (e.g. yeast cells) as well as higher eukaryotic cells. The host cell can then be cultured under appropriate conditions, whereby the recombinant lectin is expressed. The recombinant lectin can thus be obtained by isolation as an expression product from the host cell. Recombinant proteins can be purified by conventional techniques known in the art, typically conventional chromatographic methods.

In some embodiments, the recombinant Sclerotium rolfsii lectin is for use in the treatment or prevention of infectious disease caused by Coronaviridae wherein the infectious disease caused by Coronaviridae is selected from but not limited to SARS-COV2, SARS-COV, MERS-COV, HCoV-229E, HCoV-OC43, HCoV-NL63, HCoV-HKU1, and mutants thereof.

In some embodiments, the recombinant Sclerotium rolfsii lectin is for use in the treatment or prevention of infectious disease caused by Coronaviridae wherein the infectious disease caused by Coronaviridae is selected from SARS-COV2 and mutants thereof.

In some embodiments, the recombinant Sclerotium rolfsii lectin is capable of inhibiting or reducing the viral entry by blocking SpikeS1 protein of SARS-COV2 with ACE2. The recombinant Sclerotium rolfsii lectin interrupts SARS-COV-2 entry in cell via AT1R downregulation and significantly inhibits relevant proteases in pathogenesis of Covid.

In some embodiments, the recombinant Sclerotium rolfsii lectin is capable of inhibiting or modulating the cytokine storm induced by mutant SpikeS1 proteins (SARS-COV-2 derived) in lung and immune cells. The recombinant Sclerotium rolfsii lectin of SEQ ID NO: 2 significantly inhibits cytokines such as IL6 and IL8 as compared to control, in lung cells (A549) and immune cells (THP-1).

As is known in the art, cytokines are small proteins involved in cell signaling. Cytokines include chemokines, interferons, interleukins, lymphokines, and tumour necrosis factors (TNF). As used herein, “Interleukin” (IL) includes IL-1, IL-2, IL-6, IL-8, IL-10, IL-12, IL-13, IL-15, IL-17, IL-18, IL-1B, IL-17A, IL-17F, IL-22, IL-26, IL-23, IL-20, and IL-15.

In one embodiment of the present invention, recombinant Sclerotium rolfsii lectin of SEQ ID NO: 2 is capable of inhibiting proteasessuch as Cathepsin L, 3CL, DPP4 relevant in COVID-19 disease. The recombinant Sclerotium rolfsii lectin of SEQ ID NO: 2 significantly inhibits proteases such as Cathepsin L, 3CL, DPP4 which plays key role in biology of Covid 19 disease.

It will be understood that the term “treatment” comprises substantially curing (i.e. eliminating) or reducing (either permanently or temporarily) the symptoms associated with the infection of SARS-COV2 or its mutants. Such symptoms may include cytokine storm.

It will be understood that the term “prevention” may comprise preventing the onset of the infection of SARS-COV2 or its mutants, or preventing or slowing the progression of the disease caused by SARS-COV2 or its mutants.

The treatment or prevention may comprise administering a therapeutically effective amount of the recombinant Sclerotium rolfsii lectin to the subject. In some embodiments, the recombinant Sclerotium rolfsii lectin is administered at a dose of from 0.001 to 1000 mg/kg, from 0.5 to 100 mg/kg or from 1 to 50 mg/kg. It will be within the capabilities of the skilled person to determine an amount of lectin protein to be administered according to the nature of the condition being treated and the subject.

In some embodiments the recombinant Sclerotium rolfsii lectin is administered in combination with a further therapeutic agent.

In some embodiments, the present invention thus also provides a pharmaceutical composition comprising a recombinant Sclerotium rolfsii lectin and a pharmaceutically acceptable diluent or excipient. Optionally, the pharmaceutical composition may comprise a further therapeutic agent.

In some embodiments, the present invention further provides a method of treatment or prevention of disease caused by SARS-COV2 or its mutants in a subject, the method comprising administering a recombinant Sclerotium rolfsii lectin or a pharmaceutical composition as described herein to the subject.

Administration of the recombinant Sclerotium rolfsii lectin or composition may be by any suitable route, including but not limited to, injection (including intravenous (bolus or infusion), intra-arterial, intraperitoneal, subcutaneous (bolus or infusion), intraventricular, intramuscular, or subarachnoidal), oral ingestion (e.g. of a tablet, gel, lozenge or liquid), inhalation, topical, via a mucosa (such as the oral, nasal or rectal mucosa), by delivery in the form of a spray, tablet, transdermal patch, subcutaneous implant or in the form of a suppository.

In some embodiments, recombinant Sclerotium rolfsii lectin (such as a recombinant lectin having the amino acid sequence of SEQ ID NO: 2 or a pharmaceutical composition as described herein isadministered to the subject enterally, parenterally or topically. The lectin or pharmaceutical composition may be administered as a dosage form which is solid (such as tablet or capsule), a lyophilized powder, a liquid (such as solution or suspension), a semi-solid, aerosol spray or any other form as known to the person skilled in the art. The lectin or the pharmaceutical composition may be administered to the subject by injecting a solution or suspension intravenously, intramuscularly, intraperitoneally, subcutaneously, or intradermally, by depot injection, or it may be administered intrathecally, transdermally, sublingually or by oral, topical or inhalation methods.

The subject may be a mammalian subject. In some embodiments, the subject is human. In particular, the subject may be a human subject suffering from or seeking prevention from SARS-CoV2 or its mutants.

The present invention relates to the treatment of disease caused by SARS-COV2 or its mutantsusing a recombinant Sclerotium rolfsii lectin having the amino acid sequence of SEQ ID NO: 2. The invention also relates to a method for the prevention of, or onset of or progression of disease caused by SARS-COV2 its mutantsin the subject using a lectin, such as of SEQ ID NO: 2. It further relates to a method to cure or reduce the effect of SARS-COV2 in the subject using a recombinant Sclerotium rolfsii lectin, such as of SEQ ID NO: 2.

In one embodiment of the present invention, the effect of recombinant Sclerotium rolfsii lectin of SEQ ID NO: 2 on binding inhibition of SpikeS1 protein with ACE2 was assessed. SARS-COV2 enters the human body via SpikeS1 protein that binds to ACE2 receptors present on cells of the lung cells. Inhibition of binding of viral SpikeS1 protein and ACE2 protein has been widely considered as a preventive strategy for COVID-19. Varying concentrations of recombinant Sclerotium rolfsii lectin of SEQ ID NO: 2 and positive control Emodin along with ACE2 inhibitor Screening reagent were added in a 96-well plate (pre-coated with Rabbit Fc-tagged SARS-Cov-2 Spike SI RBD) and incubated and measured absorbance using a spectrophotometer at 450 nm wavelength. Results demonstrated that recombinant lectin protein of SEQ ID NO: 2 (0.01 μg/ml-0.5 μg/ml) demonstrated 46.30%-62.7 0% inhibition of binding of SpikeS1 protein with ACE2 protein as compared to positive control Emodin (0.1 μg/ml-50 μg/ml) which demonstrated 38.9%-62.8% inhibition of binding of SpikeS1 protein with ACE2. It is evident from the above result that recombinant Sclerotium rolfsii lectin of SEQ ID NO: 2 inhibits binding of SpikeS 1 and ACE2 protein which may interrupt SARS-COV-2 entry in cell via AT1R downregulation thereby preventing pathogenesis of the SARS-COV2.

In one embodiment of the present invention, the inhibitory effect of recombinant Sclerotium rolfsii lectin of SEQ ID NO: 2 on mutant SpikeS1 proteins (SARS-COV-2 derived) induced cytokine storm in lung and immune cells was evaluated. Human lung epithelial cells (A549) and immune cells (THP-1) were treated with recombinant Sclerotium rolfsii lectin of SEQ ID NO: 2 and mutant SpikeS1 proteins (SARS-COV-2 derived) for 48 h. Levels of cytokines were determined in supernatants and inhibitory effect of recombinant Sclerotium rolfsii lectin of SEQ ID NO: 2 on cytokine expression was measured by ELISA. Studies revealed that Significant inhibition of inflammatory cytokines induced by mutant SARS-COV-2 spike protein S1 in immune cells (percentage inhibition of IL6 is in the range of 9%-56% and IL8 in the range of 29%-54% and lung cells (percentage inhibition of IL6 is in the range of 65%-85% and IL8 in the range of 11%-29%) indicate the anti-inflammatory potential of recombinant Sclerotium rolfsii lectin of SEQ ID NO: 2. This strengthens the role of recombinant Sclerotium rolfsii lectin of SEQ ID NO: 2 in curbing the cytokine storm and hyper-inflammatory state in Covid-19.

In one embodiment of the present invention, the inhibitory effect of recombinant Sclerotium rolfsii lectin of SEQ ID NO: 2 on proteases relevant in COVID-19 disease is further evaluated using cell free assay. Proteases such as Cathepsin L, 3CL and DPP4 play key role in COVID-19 infection. Cathepsin mediates the cleavage of the S1 subunit of the coronavirus surface spike glycoprotein and thus facilitating coronavirus entry into human host cells, virus and host cell endosome membrane fusion, and viral RNA release for next round of replication. 3C-like protease (3CLpro) is essential for SARS-COV replication. DPP4 interacts with spike glycoprotein Slb domain to promote virus entry. Purified proteases were incubated with recombinant Sclerotium rolfsii lectin of SEQ ID NO: 2 and corresponding fluorogenic substrates were used to evaluate the inhibitory effect. Recombinant Sclerotium rolfsii lectin of SEQ ID NO: 2 demonstrated significant inhibition of Cathepsin L (71.6%) and 3CL (26.2%), moderate inhibition of DPP4 (16.7%).

### EXAMPLES

The following examples are given to demonstrate the best mode of performance of the invention. Examples do not limit the invention in any manner.

Formulation of recombinant Sclerotium Rolfsii Lectin of SEQ ID NO: 2

The recombinant Sclerotium rolfsii lectin of SEQ ID NO: 2 for injection was provided as lyophilized cake in 2.5 mg quantity, which was dissolved in 1.2 mL of water for injection to give final concentration of 2.5 mg/mL

### ABBREVIATIONS USED

| ABBREVIATION | DESCRIPTION |
|---|---|
| CO₂ | Carbon Dioxide |
| ° C | Degree Centigrade |
| DPP4 | Dipeptidyl-peptidase 4 |
| μg | Microgram |
| μl | Microliter |
| μM | Micromolar |
| h | hours |
| ml | Milliliter |
| min | Minutes |
| MIDS | Material Information Detail Sheet |
| nm | Nanometer |
| % | Percentage |
| RNA | Ribonucleic acid |
| SFM | Serum Free Medium |
| SARS-COV2 | Severe Acute Respiratory Syndrome Coronavirus 2 |
| DMSO | Dimethyl sulfoxide |
| NaCl | Sodium Chloride |
| Na₂HPO₄ | Disodium phosphate |
| KH₂PO₄ | Monopotassium phosphate |
| KCl | Potassium chloride |
| DMEM | Dulbecco's Modified Eagle's Medium |
| RPMI | Roswell Park Memorial Institute |
| ELISA | Enzyme- Linked Immunosorbent Assay |
| H | Hour |
| IFN-γ | Interferon -γ |
| IL-6 | Interleukin-6 |
| IL-8 | Interleukin-8 |
| KDa | KiloDalton |

### Example 1: Effect of Recombinant Sclerotium rolfsii Lectin of SEQ ID NO: 2 on Inhibition of Binding of SpikeS1 Protein with ACE2 Protein

Effect of recombinant Sclerotium rolfsii lectin of SEQ ID NO: 2 on binding of SpikeS1 protein to ACE2 was evaluated using SARS-COV-2 Spike-ACE2 Interaction Inhibitor Screening Assay kit. In 96 well plate (pre-coated with Rabbit Fc-tagged SARS-Cov-2 Spike S1 RBD) 50 μL immunoassay buffer (provided in kit), 50 μL of different concentrations of recombinant Sclerotium rolfsii lectin of SEQ ID NO: 2, positive control and 50 μL ACE2 Inhibitor Screening reagent were added and incubated for 30 mins at RT. After incubation, 50 μL Spike Inhibitor screening reagent was added to the wells and incubated for 30 mins at RT. Post incubation, the sample mixture was removed, and plate was washed 5 times with Wash Buffer (1×). 150 μL Anti-His-HRP Conjugate (1×) was added to the wells and the plate was incubated for 30 minutes at RT on an orbital shaker. After incubation, the plate was washed 5 times with Wash Buffer (1×). 175 μL TMB Substrate was added to the plate and incubated for 20 minutes. The color in the wells turn to medium-to-dark blue. The reaction was stopped using stop buffer and absorbance of each well was read at 450 nm using synergy HT micro plate reader. % inhibition was calculated using the following formula:

$$\% Inhibition = \frac{( OD of Control - OD of well with TI / PC ) \times 100}{OD of Control}$$

TABLE 1
Effect of recombinant Sclerotium rolfsii lectin of SEQ ID NO:
2 on inhibition of binding of SpikeS1 protein with ACE2 protein

| | | Inhibition of SpikeS1- |
|---|---|---|
| Sample | Concentration | ACE2 binding (%) |
| Emodin (PC) (μg/ml) | 0.1 | 38.9 |
| | 1 | 44.3 |
| | 10 | 48.9 |
| | 50 | 62.8 |
| recombinantSclerotium | 0.01 | 46.3 |
| rolfsii lectin of SEQ ID | 0.1 | 52.1 |
| NO: 2(mg/ml) | 0.25 | 59.4 |
| | 0.5 | 62.7 |

The results showed that recombinant Sclerotium rolfsii lectin of SEQ ID NO: 2 for injection demonstrated 46.3%-62.7% inhibition of SpikeS1-ACE2 binding as compared to control.

### Example 2: Determination of Non-Cytotoxic Doses of Recombinant Sclerotiumrolfsii Lectin of SEQ ID NO: 2

A549 cells were plated in DMEM+10% FBS at a density of 10,000 cells/well in 96-well culture plates and incubated at 37° C. for 24 h. THP-1 cells were plated in RPMI+10% FBS at a density of 10,000 cells/well in 96-well culture plates and incubated at 37° C. for 24 h. Cells were sera-starved for 24 h. Next day, cells were treated with recombinant Sclerotium rolfsii lectin of SEQ ID NO: 2 at different concentrations ranging from 10 ng/ml-50 μg/ml. Untreated cells were used as control. Cells treated with dexamethasone and hydroxychloroquine were included as positive control. After treatment, cells were incubated in a 5% CO2 incubator for 48 h. Effect of recombinant Sclerotium rolfsii lectin of SEQ ID NO: 2 on cell viability was estimated by 3-(4, 5-dimethythiazol-2-yl)-2,5-diphenyl tetrazolium bromide (MTT) assay.

TABLE 2
Effect of recombinant Sclerotium rolfsii lectin of SEQ
ID NO: 2 on viability of Immune (THP-1) cells after 48 h
Immune cells (THP-1)

| Sample | Concentration | | % Cytotoxicity | % Viability |
|---|---|---|---|---|
| Dexamethasone | 0.1 | μM | −10.0 | 110.0 |
| (PC-1) | 1 | μM | 1.8 | 98.2 |
| | 5 | μM | 16.9 | 83.1 |
| | 10 | μM | 4.4 | 95.6 |
| | 20 | μM | −4.3 | 104.3 |
| Hydroxychloroquine | 0.1 | μM | −13.4 | 113.4 |
| sulphate (PC-2) | 1 | μM | −5.2 | 105.2 |
| | 5 | μM | 4.2 | 95.8 |
| | 10 | μM | −4.8 | 104.8 |
| | 20 | μM | 10.7 | 89.3 |
| Recombinant | 10 | ng/ml | 12.9 | 87.1 |
| Sclerotium Rolfsii | 100 | ng/ml | 8.9 | 91.1 |
| Lectin of Seq | 1 | μg/ml | 25.7 | 74.3 |
| Id No: 2 | 10 | μg/ml | 19.1 | 80.9 |
| | 50 | μg/ml | 27.5 | 72.5 |

TABLE 3
Effect of recombinant Sclerotium rolfsii lectin of SEQ
ID NO: 2 on viability of Lung (A549) cells after 48 h
Lung cells (A549)

| Sample | Concentration | | % Cytotoxicity | % Viability |
|---|---|---|---|---|
| Untreated | | | 0 | 0 |
| Dexamethasone | 0.1 | μM | −22.7 | 122.7 |
| (PC-1) | 1 | μM | 23.7 | 76.3 |
| | 5 | μM | 10.0 | 90.0 |
| | 10 | μM | 17.4 | 82.6 |
| | 20 | μM | 30.5 | 69.5 |
| Hydroxychloroquine | 0.1 | μM | 21.0 | 79.0 |
| sulphate (PC-2) | 1 | μM | −5.2 | 105.2 |
| | 5 | μM | −1.0 | 101.0 |
| | 10 | μM | −10.9 | 110.9 |
| | 20 | μM | 27.6 | 72.4 |
| Recombinant | 10 | ng/ml | 1.0 | 99.0 |
| Sclerotium Rolfsii | 100 | ng/ml | −18.3 | 118.3 |
| Lectin of Seq Id | 1 | μg/ml | 4.4 | 95.6 |
| No: 2 | 10 | μg/ml | 10.4 | 89.6 |
| | 50 | μg/ml | 17.9 | 82.1 |

Recombinant Sclerotium rolfsii lectin of SEQ ID NO: 2 in the concentration range of 10 ng/ml-50 μg/ml demonstrated more than >70% cell viability in A549 and THP-1 cells as compared to control.

### Example 2.1 Determination of Cytokines Levels by ELISA

A549 and THP-1 cells were counted using hemocytometer and plated in a tissue culture plate at the density of 0.1×106 cells/well in 24 well plate in growth medium supplemented with 10% FBS. The above cells were incubated at 37° C. in 5% CO2 incubator for 24 h. Cells were sera starved for 24 h. Cells were treated with Recombinant Sclerotium rolfsii lectin of SEQ ID NO: 2; Recombinant Sclerotium rolfsii lectin of SEQ ID NO: 2 at different concentrations ranging from 10 ng/ml-50 μg/ml and stimulated with mutant SpikeS1 proteins (SARS-COV-2 derived) for 48 h. Cells treated with Dexamethasone and Hydroxychloroquine and stimulated with mutant SpikeS1 protein (SARS-COV-2 derived) were included as positive controls. Cells stimulated with mutant SpikeS1 protein (SARS-COV-2 derived) alone were included as control. Untreated cells were included as Negative control. After 48 h, cell culture supernatants were collected from each well and stored at −20° C. till analysis. The levels of inflammatory markers were determined by ELISA as follows: Assay diluent was added to each well. Respective kit standards and samples (supernatants of cells) were directly pipetted into the wells and incubated for 2 h at RT. After washing away any unbound substances for a total of 5 times, respective conjugate was added to each well and incubated for 1-2 h at RT. Following a wash (5 times) to remove any unbound conjugate, substrate solution was added to the wells and incubated for 30 min at RT in dark. The reaction was stopped by adding Stop solution to each well.

The optical density of the color was measured at 450 nm. The percent inhibition in each sample was determined as follows:

[{Concentration of Cytokines (pg/ml) in Control cells (stimulated with mutant spike protein alone)−Concentration of Cytokines (pg/ml) in Test Item+Mutant Spike protein treated cells}/Concentration of Cytokines (pg/ml) in Control cells (stimulated with mutant spike protein alone)]*100.

TABLE 4
Inhibitory effect of Recombinant Sclerotium rolfsii lectin
of SEQ ID NO: 2 on levels of cytokines induced by mutant SARS-
COV-2-spike S1 (E484K) protein in Immune cells (THP-1)

| | | | | Inhibition of | |
|---|---|---|---|---|---|
| Sample | | Concentration | | IL-6 | IL-8 |
| Untreated | | | | 0.0 | |
| Mutant Spike (E484K) 5 μg/ml | | | | 0.0 | |
| Mutant Spike | Dexamethasone (PC-1) | 0.1 | | 86.1 | 56.0 |
| (E484K) − | (μM) | 1 | | 100.0 | 57.9 |
| 5 μg/ml+ | | 5 | | 92.4 | 57.3 |
| | | 10 | | 99.2 | 43.4 |
| | | 20 | | 100.0 | 31.4 |
| | Hydroxychloroquine | 0.1 | | 90.7 | 23.2 |
| | sulphate (PC-2) | 1 | | 83.9 | 36.8 |
| | (μM) | 5 | | 94.2 | 53.4 |
| | | 10 | | 84.5 | 53.5 |
| | | 20 | | 100.0 | 36.1 |
| | recombinantSclerotium | 10 | ng/ml | 85.7 | 12.9 |
| | rolfsii lectin of SEQ ID | 100 | ng/ml | 79.9 | 28.9 |
| | NO: 2(mg/ml) | 1 | μg/ml | 72.8 | 21.7 |
| | | 10 | μg/ml | 74.6 | 13.0 |
| | | 50 | μg/ml | 65.7 | 10.9 |

Recombinant Sclerotium rolfsii lectin of SEQ ID NO: 2 for Injection (10 ng/ml-50 μg/ml) demonstrated significant (p<0.001) inhibition of IL-8 and IL-6 as compared to mutant SARS-COV-2-spike S1 protein (E484K) induced levels.

TABLE 5
Inhibitory effect of Recombinant Sclerotium rolfsii lectin
of SEQ ID NO: 2 for Injection on levels of cytokines induced by
mutant SARS-COV-2-spike S1 (N501Y) protein in Lung cells (A549)

| | | | | Inhibition of | |
|---|---|---|---|---|---|
| Sample | | Concentration | | IL-6 | IL-8 |
| Untreated | | | | 0.0 | |
| N501Y- 10 μg/ml | | | | 0.0 | |
| Mutant Spike | Dexamethasone (PC-1) | 0.1 | | 76.5 | 77.8 |
| (N501Y) − | (μM) | 1 | | 83.0 | 71.9 |
| 10 μg/ml+ | | 5 | | 71.3 | 71.6 |
| | | 10 | | 85.8 | 65.5 |
| | | 20 | | 80.0 | 70.8 |
| | Hydroxychloroquine | 0.1 | | 34.7 | 54.4 |
| | sulphate (PC-2) | 1 | | 53.4 | 40.8 |
| | (μM) | 5 | | 66.5 | 56.7 |
| | | 10 | | 60.7 | 44.0 |
| | | 20 | | 71.4 | 65.0 |
| | recombinantSclerotium | 10 | ng/ml | 54.7 | 54.2 |
| | rolfsii lectin of SEQ ID | 100 | ng/ml | 32.2 | 29.2 |
| | NO: 2(mg/ml) | 1 | μg/ml | 56.7 | 30.7 |
| | | 10 | μg/ml | 9.0 | 49.2 |
| | | 50 | μg/ml | 26.6 | 30.4 |

Recombinant Sclerotium rolfsii lectin of SEQ ID NO: 2 (10 ng/ml-50 μg/ml) demonstrated significant (p<0.001) inhibition of IL-8 and IL-6 as compared to mutant SARS-COV-2-spike S1 protein (N501Y) induced levels.

### Example 3: Evaluation of Inhibitory Effect of Recombinant Sclerotium rolfsii Lectin of SEQ ID NO: 2 on Proteases Relevant in Covid-19 Using Cell Free Assays

Inhibitory effect of Recombinant Sclerotium rolfsii lectin of SEQ ID NO: 2 on different proteases relevant in Covid-19 wasstudied using specific cell-free, enzymes-based assay kits.

### CATHEPSIN L Inhibitor Screening Assay

Reagents were added as below in the same order as mentioned:

| Component | Enzyme | | Positive | | Test | | Blank | |
|---|---|---|---|---|---|---|---|---|
| Enzyme solution (0.02 ng/μl | 20 | μl | 20 | μl | 20 | μl | — | |
| Cathepsin L) | | | | | | | | |
| 1X Cathepsin buffer | — | | — | | — | | 20 | μl |
| Inhibitor (in Cathepsin buffer) | — | | — | | 5 | μl | — | |
| Inhibitor Buffer (No inhibitor) | 5 | μl | — | | — | | 5 | μl |
| E-64 (positive control provided | — | | 5 | μl | | | — | |
| within the kit) | | | | | | | | |
| Substrate solution (10 μM) | 25 | μl | 25 | μl | 25 | μl | 25 | μl |
| Total Volume | 50 | μl | 50 | μl | 50 | μl | 50 | μl |

After the addition of substrate solution, reaction mix was incubated at room temperature for 60 minutes.
Fluorescence was read at (excitation=360 nm; emission=460 nm) in microplate reader. Decrease in fluorescence indicated inhibition of the Cathepsin L.
Inhibition of Cathepsin L was calculated as:

$$\% Inhibition = [ ( {RFU}_{Enzyme {control}^{-}} {RFU}_{TI / PC} ) / {RFU}_{Enzyme control} ] * 100$$

TABLE 6
Effect of recombinant Sclerotium rolfsi lectin
of SEQ ID NO: 2 on inhibition of Cathepsin L

| | | |
|---|---|---|
| | | % Inhibition wrt |
| Sample | Concentration | Enzyme Control |

Blank
Enzyme Control

| | | |
|---|---|---|
| E-64 (PC) (μM) | 0.0001 | 7.4 |
| | 0.01 | 24.2 |
| | 0.1 | 61.0 |
| recombinantSclerotium | 10 | −4.1 |
| rolfsii lectin of SEQ ID | 25 | 31.4 |
| NO: 2(μg/ml) | 50 | 60.0 |
| | 100 | 63.7 |
| | 250 | 71.6 |

Conclusion Recombinant Sclerotium rolfsii lectin of SEQ ID NO: 2 (10 μg/ml-250 μg/ml) demonstrated 31.4%-71.6% significant inhibition (p<0.001) of Cathepsin L as compared to enzyme control.

### 3CL Protease, (SARSCOV-2) Assay

Reagents were added as below in the same order as mentioned:

| Component | Enzyme | Positive | Test | Blank |
|---|---|---|---|---|
| 3CL Protease (3-5 ng/μl) | 30 μl | 30 μl | 30 μl | — |
| Assay Buffer (with DTT) | — | — | — | 30 μl |
| Test Inhibitor | — | — | 10 μl | — |
| Inhibitor Buffer (no inhibitor) | 10 μl | — | — | 10 μl |
| GC376 (500 μM) (positive | — | 10 μl | | — |
| control provided within | | | | |
| Substrate solution (10 μM) | 10 μl | 10 μl | 10 μl | 10 μl |
| Total Volume | 50 μl | 50 μl | 50 μl | 50 μl |

After the addition of substrate solution, reaction mix was incubated at room temperature for overnight.
Fluorescence was read at (excitation=360 nm; emission=460 nm) in microplate reader. Decrease in fluorescence indicated inhibition of the 3CL.
Inhibition of 3CL was calculated as:

$$\% Inhibition = [ ( {RFU}_{Enzyme {control}^{-}} {RFU}_{TI / PC} ) / {RFU}_{Enzyme control} ] * 100$$

TABLE 7
Effect of recombinant Sclerotium rolfsii lectin of
SEQ ID NO: 2 for Injection on inhibition of 3CL Protease

| | | | % Inhibition wrt |
|---|---|---|---|
| | Sample | Concentration | Enzyme Control |
| Blank | | | |
| Enzyme Control | | | 0 |
| | GC376 (PC) (μM) | 0.1 | 25.3 |
| | | 1 | 72.1 |
| | | 10 | 96.1 |
| | | 100 | 94.4 |
| | recombinantSclerotium | 10 | 11.3 |
| | rolfsii lectin of SEQ ID | 50 | 19.3 |
| | NO: 2(μg/ml) | 100 | 22.5 |
| | | 250 | 26.2 |
| | | 500 | 17.4 |

Conclusion: Recombinant Sclerotium rolfsii lectin of SEQ ID NO: 2 (10 μg/ml-500 μg/ml) demonstrated 11.3%-26.2% significant inhibition (p<0.001) of 3CL as compared to enzyme control.

### DPP4 Assay

Reagents were added as below in the same order as mentioned:

| Component | Enzyme | | Positive | | Test | | Blank | |
|---|---|---|---|---|---|---|---|---|
| DPP assay buffer | 80 | μl | 80 | μl | 80 | μl | 90 | μl |
| DPP substrate 1 | 5 | μl | 5 | μl | 5 | μl | 5 | μl |
| (100 μM) | | | | | | | | |
| Test Inhibitor | — | | — | | 5 | μl | — | |
| Sitagliptin | — | | 5 | μl | | | — | |
| Inhibitor Buffer (no | 5 | μl | | | | | 5 | μl |
| DPP4 (0.1 ng/μl) | 10 | μl | 10 | μl | 10 | μl | — | |
| Total Volume | 100 | μl | 100 | μl | 100 | μl | 100 | μl |

After the addition of substrate solution, reaction mix was incubated at room temperature for 10 minutes.
Fluorescence was read at (excitation=360 nm; emission=460 nm) in microplate reader. Decrease in fluorescence indicated inhibition of the DPP4.
Inhibition of DPP4 was calculated as:

$$\% Inhibition = [ ( {RFU}_{Enzyme {control}^{-}} {RFU}_{TI / PC} ) / {RFU}_{Enzyme control} ] * 100$$

TABLE 8
Effect of recombinant Sclerotium rolfsii
lectin of SEQ ID NO: 2 on inhibition of DPP4

| | | | % Inhibition wrt |
|---|---|---|---|
| | Sample | Concentration | Enzyme Control |
| Blank | | | |
| Enzyme Control | | | 0 |
| | GC376 (PC) (μM) | 0.001 | 32.4 |
| | | 0.01 | 76.2 |
| | | 0.1 | 87.1 |
| | | 1 | 99.5 |
| | recombinantSclerotium | 10 | 7.1 |
| | rolfsii lectin of SEQ ID | 25 | 16.7 |
| | NO: 2(μg/ml) | 50 | 4.6 |
| | | 100 | 9.5 |
| | | 125 | 11.4 |

Conclusion: Recombinant Sclerotium rolfsii lectin of SEQ ID NO: 2 (10 μg/ml-125 μg/ml) demonstrated 4.6%-16.7% inhibition of DPP4 as compared to Enzyme control.

### Example 4: Inhibitory Effect of Recombinant Sclerotium rolfsii Lectin of SEQ ID NO: 2 on SARS-Cov-2 Spike S1 Protein Induced Cytokine Storm

The present study evaluated the inhibitory effect of Recombinant Sclerotium rolfsii lectin of SEQ ID NO: 2 on SARS-Cov-2 Spike S1 protein induced cytokine storm in Bladder, Heart, Colon, Vascular/Blood vessels, Kidney, Bone, Liver and Pancreas cell lines. Cells were counted using hemocytometer and plated in a 48 well tissue culture plate in growth medium supplemented with 10% FBS. Cells were incubated at 37° C. in 5% CO2 incubator for 24 h after whichthe cells were sera starved for 24 h. Cells were then treated with Recombinant Sclerotium rolfsii lectin of SEQ ID NO: 2 for injection at different concentrations for 24 h and then stimulated with SARS-COV-2-spike S1 protein for 24 h. Stimulation of cells with SARS-COV-2-spike S1 protein resulted in significantly elevated levels of inflammatory cytokines as compared to untreated levels. Cells treated with dexamethasone and hydroxychloroquine stimulated with SARS-COV-2-spike S1 protein were included as positive controls. Cells stimulated with SARS-COV-2-spike S1 protein alone were included as control. Untreated cells were included as negative control. After 24 h, cell culture supernatants were collected from each well and stored at 20 degree Celsius till analysis. The levels of inflammatory markers were determined by ELISA. The percent inhibition in each sample was determined as follows:

$$[ \{ Concentration of cytokines ( pg / ml ) in control cells induced with Spike protein alone - Concentration of cytokines ( pg / ml ) in test item treated cells stimulated with Spike protein ) \} / Conocentration of cytokines ( pg / ml ) in control cells induced with Spike protein alone ] * 100$$

TABLE 9
Inhibitory effect of Recombinant Sclerotium rolfsii lectin of SEQ ID
NO: 2 on expression of inflammatory markers in Bladder cells (T24)induced
by SpikeS1 protein (SARS-COV-2)
Bladder Cells

| | | | | Conc. of | % | Con. of | % |
|---|---|---|---|---|---|---|---|
| | | | | IL-6 | Inhibition | IL-8 | Inhibition |
| SAMPLE | | | | [symbol] | [symbol] | [symbol] | [symbol] |
| Untreated | | | | 235.9 | | 1980.9 | |
| Spike S1 Protein (5 μg/ml) | | | | 577.6 | 0.0 | 4273.8 | 0.0 |
| Spike S1 | Dexamethasone | 0.1 | μM | 292.2 | 49.4 | 2458.5 | 42.5 |
| Protein | (PC-1) | 1 | μM | 348.5 | 39.7 | 2008.8 | 53.0 |
| (5 μg/ml) + | | 10 | μM | 369.4 | 36.0 | 2198.1 | 48.6 |
| | | 20 | μM | 365.4 | 36.7 | 2386.4 | 44.2 |
| | Hydroxychloroquine | 0.1 | μM | 432.0 | 25.2 | 2661.3 | 37.7 |
| | sulphate | 1 | μM | 410.8 | 28.9 | 2527.7 | 40.9 |
| | (PC-2) | 10 | μM | 430.2 | 25.5 | 2679.5 | 37.3 |
| | | 20 | μM | 415.8 | 28.0 | 2470.0 | 42.2 |
| | SEQ ID: 2 | 10 | ng/ml | 443.5 | 23.2 | 3005.3 | 29.7 |
| | | 100 | ng/ml | 455.1 | 21.2 | 2864.0 | 33.0 |
| | | 1 | μg/ml | 505.1 | 12.5 | 3402.2 | 20.4 |
| | | 10 | μg/ml | 505.5 | 12.5 | 3300.3 | 22.8 |

[symbol]

indicates data missing or illegible when filed

In bladder cells (T24), recombinant Sclerotium rolfsii lectin of SEQ ID 2 (10 ng/ml 10 μg/ml) demonstrated significant (p<0.001) inhibition of inflammatory cytokinesIL-6 and IL-8 levels as compared to levels induced by Spike S1 protein (SARS-COV-2).

TABLE 10
Inhibitory effect of Recombinant Sclerotium rolfsii lectin of SEQ ID
NO: 2 on expression of inflammatory markers in Cardiac cells (H9C2)induced
by SpikeS1 protein (SARS-COV-2)
Cardiac Cells

| | | | | Conc. | % | Con. of | % |
|---|---|---|---|---|---|---|---|
| SAMPLE | | | | of IL-6 | Inhibition | IL-8 | Inhibition |
| Untreated | | | | 126.2 | | 263.4 | |
| Spike S1 Protein (5 μg/ml) | | | | 505.7 | 0.0 | 1431.8 | 0.0 |
| Spike | | 0.1 | μM | 50.8 | 90.0 | 31.2 | 97.8 |
| S1 | | 1 | μM | 44.5 | 91.2 | 18.6 | 98.7 |
| Protein | Dexamethasone | 10 | μM | 53.3 | 89.5 | 29.1 | 98.0 |
| (5 μg/ml) + | (PC-1) | 20 | μM | 51.8 | 89.7 | 48.7 | 96.6 |
| | Hydroxychloroquine | 0.1 | μM | 420.1 | 16.9 | 952.1 | 33.5 |
| | sulphate | 1 | μM | 313.4 | 38.0 | 768.5 | 46.3 |
| | (PC-2) | 10 | μM | 125.2 | 75.2 | 231.8 | 83.8 |
| | | 20 | μM | 61.8 | 87.8 | 45.5 | 96.8 |
| | SEQ ID: 2 | 10 | ng/ml | 458.7 | 9.3 | 1268.2 | 11.4 |
| | | 100 | ng/ml | 368.0 | 27.2 | 853.4 | 40.4 |
| | | 1 | μg/ml | 303.3 | 40.0 | 648.1 | 54.7 |
| | | 10 | μg/ml | 186.4 | 63.1 | 329.9 | 77.0 |
| | | 50 | μg/ml | 198.4 | 60.8 | 342.1 | 76.1 |

In Cardiac cells (119C2), recombinant Sclerotium rolfsii lectin of SEQ ID 2 for Injection (10 ng/ml-50 μg/ml) demonstrated significant (p<0.001) inhibition of inflammatory cytokines, IL-6 and IL-8 levels as compared to levels induced by Spike S1 protein (SARS-COV-2).

TABLE 11
Inhibitory effect of Recombinant Sclerotium rolfsii lectin of SEQ ID
NO: 2 on expression of inflammatory markers in Colon cells (HT-29) induced
by SpikeS1 protein (SARS-COV-2)
Colon Cells

| | | | | | % | | % |
|---|---|---|---|---|---|---|---|
| | | | | Conc. | Inhibition | Con. of | Inhibition |
| | | | | of IL-6 | (wrt | IL-8 | (wrt |
| SAMPLE | | | | (pg/ml) | Control) | (pg/ml) | Control) |
| Untreated | | | | 81.5 | | 19.6 | |
| Spike S1 Protein (1 μg/ml) | | | | 235.8 | 0.0 | 670.6 | 0.0 |
| Spike | | 0.1 | μM | 106.0 | 55.0 | 44.1 | 93.4 |
| S1 | | 1 | μM | 89.1 | 62.2 | 18.2 | 97.3 |
| Protein | Dexamethasone | 10 | μM | 73.6 | 68.8 | 53.5 | 92.0 |
| (1 μg/ml) + | (PC-1) | 20 | μM | 109.0 | 53.8 | 66.4 | 90.1 |
| | Hydroxychloroquine | 0.1 | μM | 114.8 | 51.3 | 49.1 | 92.7 |
| | sulphate | 1 | μM | 91.1 | 61.4 | 92.3 | 86.2 |
| | (PC-2) | 10 | μM | 125.2 | 46.9 | 99.5 | 85.2 |
| | | 20 | μM | 104.2 | 55.8 | 37.6 | 94.4 |
| | SEQ ID: 2 | 10 | ng/ml | 87.3 | 63.0 | 61.4 | 90.8 |
| | | 100 | ng/ml | 83.2 | 64.7 | 31.9 | 95.2 |
| | | 1 | μg/ml | 53.1 | 77.5 | 76.5 | 88.6 |
| | | 10 | μg/ml | 78.4 | 66.8 | 34.0 | 94.9 |
| | | 50 | μg/ml | 122.1 | 48.2 | 31.9 | 95.2 |

In Colon cells (HT-29), recombinant Sclerotium rolfsii lectin of SEQ ID 2 (10 ng/ml-50 μg/ml) demonstrated significant (p<0.001) inhibition of inflammatory cytokines: IL-6 and IL-8 levels as compared to levels induced by Spike S1 protein (SARS-COV-2).

TABLE 12
Inhibitory effect of Recombinant Sclerotium rolfsii lectin of SEQ ID
NO: 2 on expression of inflammatory markers in Endothelial cells (EA.hy926)
cells induced by SpikeS1 protein (SARS-COV-2)

| | | | | | % | | % |
|---|---|---|---|---|---|---|---|
| | | | | Conc. | Inhibition | Con. of | Inhibition |
| | | | | of IL-6 | (wrt | IL-8 | (wrt |
| SAMPLE | | | | (pg/ml) | [symbol] | (pg/ml) | [symbol] |
| Untreated | | | | 98.7 | | 623.8 | |
| Spike S1 Protein (1 μg/ml) | | | | 337.0 | 0.0 | 1615.7 | 0.0 |
| Spike | | 0.1 | μM | 48.1 | 85.7 | 1208.6 | 25.2 |
| S1 | | 1 | μM | 42.9 | 87.3 | 1250.3 | 22.6 |
| Protein | Dexamethasone | 10 | μM | 53.9 | 84.0 | 1633.7 | 0.0 |
| (1 μg/ml) + | (PC-1) | 20 | μM | 72.9 | 78.4 | 1766.1 | 0.0 |
| | Hydroxychloroquine | 0.1 | μM | 145.1 | 56.9 | 826.7 | 48.8 |
| | sulphate | 1 | μM | 200.3 | 40.5 | 1026.6 | 36.5 |
| | (PC-2) | 10 | μM | 64.0 | 81.0 | 1756.7 | 0.0 |
| | | 20 | μM | 58.9 | 82.5 | 1573.3 | 2.6 |
| | SEQ ID: 2 | 10 | ng/ml | 109.0 | 67.6 | 885.7 | 45.2 |
| | | 100 | ng/ml | 123.8 | 63.2 | 1007.2 | 37.7 |
| | | 1 | μg/ml | 128.4 | 61.9 | 928.8 | 42.5 |
| | | 10 | μg/ml | 186.0 | 44.8 | 808.0 | 50.0 |
| | | 50 | μg/ml | 268.5 | 20.3 | 477.8 | 70.4 |

[symbol]

indicates data missing or illegible when filed

In Endothelial/blood vessels cells (EA.hy926), recombinant Sclerotium rolfsii lectin of SEQ ID 2 (10 ng/ml-50 μg/ml) demonstrated significant (p<0.001) inhibition of inflammatory cytokines: IL-6 and IL-8 levels as compared to levels induced by Spike S1 protein (SARS-COV-2).

TABLE 13
Inhibitory effect of Recombinant Sclerotium rolfsii lectin of SEQ ID
NO: 2 on expression of inflammatory markers in Kidney cells (HEK-293)
cells induced by SpikeS1 protein (SARS-COV-2)
Kidney Cells

| | | | | | % | | % |
|---|---|---|---|---|---|---|---|
| | | | | Conc. | Inhibition | Con. of | Inhibition |
| | | | | of IL-6 | (wrt | IL-8 | (wrt |
| SAMPLE | | | | (pg/ml) | Control) | (pg/ml) | Control) |
| Untreated | | | | 112.7 | | 795.5 | |
| Spike S1 Protein (100 ng/ml) | | | | 260.1 | 0.0 | 1914.5 | 0.0 |
| Spike | Dexamethasone | 0.1 | μM | 64.5 | 75.2 | 447.3 | 76.6 |
| S1 | (PC-1) | 1 | μM | 65.6 | 74.8 | 453.6 | 76.3 |
| Protein | | 10 | μM | 79.6 | 69.4 | 551.8 | 71.2 |
| (100 ng/ml) + | | 20 | μM | 42.6 | 83.6 | 292.7 | 84.7 |
| | Hydroxychloroquine | 0.1 | μM | 94.2 | 63.8 | 651.8 | 66.0 |
| | sulphate | 1 | μM | 97.4 | 62.6 | 678.2 | 64.6 |
| | (PC-2) | 10 | μM | 137.7 | 47.1 | 959.1 | 49.9 |
| | | 20 | μM | 260.0 | 0.0 | 1809.1 | 5.5 |
| | SEQ ID: 2 | 10 | ng/ml | 107.7 | 58.6 | 750.9 | 60.8 |
| | | 100 | ng/ml | 45.6 | 82.5 | 310.9 | 83.8 |
| | | 1 | μg/ml | 39.0 | 85.0 | 270.9 | 85.8 |
| | | 10 | μg/ml | 131.9 | 49.3 | 921.8 | 51.9 |
| | | 50 | μg/ml | 135.1 | 48.1 | 943.6 | 50.7 |

In Kidney cells (HEK-293), recombinant Sclerotium rolfsii lectin of SEQ ID 2 (10 ng/ml-50 μg/ml) demonstrated significant (p<0.001) inhibition of inflammatory cytokines; IL-6 and IL-8 levels as compared to levels induced by Spike S1 protein (SARS-COV-2).

TABLE 14
Inhibitory effect of Recombinant Sclerotium rolfsii lectin of SEQ ID
NO: 2 on expression of inflammatory markers in Bone cells (MG-63) induced
by SpikeS1 protein (SARS-COV-2)
Bone Cells

| | | | | | % | | % |
|---|---|---|---|---|---|---|---|
| | | | | Conc. | Inhibition | Con. of | Inhibition |
| | | | | of IL-6 | (wrt | IL-8 | (wrt |
| SAMPLE | | | | (pg/ml) | Control) | (pg/ml) | Control) |
| Untreated | | | | 110.0 | | 581.7 | |
| Spike S1 Protein (10 μg/ml) | | | | 160.0 | 0.0 | 707.8 | 0.0 |
| Spike | Dexamethasone | 0.1 | μM | 49.3 | 69.2 | 407.8 | 42.4 |
| S1 | (PC-1) | 1 | μM | 50.9 | 68.2 | 355.6 | 49.8 |
| Protein | | 10 | μM | 46.2 | 71.1 | 400.6 | 43.4 |
| (10 μg/ml) + | | 20 | μM | 52.0 | 67.5 | 427.8 | 39.6 |
| | Hydroxychloroquine | 0.1 | μM | 136.5 | 14.7 | 703.3 | 0.6 |
| | sulphate | 1 | μM | 171.8 | −7.4 | 660.0 | 6.8 |
| | (PC-2) | 10 | μM | 233.8 | −46.1 | 774.4 | −9.4 |
| | | 20 | μM | 354.0 | −121.3 | 1388.3 | −96.2 |
| | SEQ ID : 2 | 10 | ng/ml | 135.1 | 15.6 | 668.9 | 5.5 |
| | | 100 | ng/ml | 123.1 | 23.1 | 635.6 | 10.2 |
| | | 1 | μg/ml | 104.0 | 35.0 | 565.0 | 20.2 |
| | | 10 | μg/ml | 105.6 | 34.0 | 556.1 | 21.4 |
| | | 50 | μg/ml | 173.5 | −8.4 | 578.3 | 18.3 |

In Bone cells (MG-63), recombinant Sclerotium rolfsii lectin of SEQ ID 2 (10 ng/ml-50 ng/ml) demonstrated significant (p<0.001) inhibition of inflammatory cytokines: IL-6 and IL-8 levels as compared to levels induced by Spike S1 protein (SARS-COV-2).

TABLE 15
Inhibitory effect of Recombinant Sclerotium rolfsii lectin
of SEQ ID NO: 2 on expression of inflammatory markers in liver
cells (HepG2) induced by SpikeS1 protein (SARS-COV-2)

| | | | | Conc. of | |
|---|---|---|---|---|---|
| | | | | IL-8 | % Inhibition |
| SAMPLE | | | | (pg/ml) | (wrt Control ) |
| Untreated | | | | 17.8 | |
| Spike S1 Protein (20 μg/ml) | | | | 498.6 | 0.0 |
| Spike S1 | Dexamethasone | 0.1 | μM | 91.4 | 81.7 |
| Protein | (PC-1) | 1 | μM | 21.5 | 95.7 |
| (20 μg/ml)+ | | 10 | μM | 64.4 | 87.1 |
| | | 20 | μM | 23.9 | 95.2 |
| | Hydroxy- | 0.1 | μM | 117.1 | 76.5 |
| | chloroquine | 1 | μM | 193.2 | 61.3 |
| | sulphate | 10 | μM | 162.5 | 67.4 |
| | (PC-2) | 20 | μM | 103.6 | 79.2 |
| | SEQ ID: 2 | 10 | ng/ml | 129.4 | 74.0 |
| | | 100 | ng/ml | 79.1 | 84.1 |
| | | 1 | μg/ml | 161.3 | 67.6 |
| | | 10 | μg/ml | 526.8 | −5.7 |
| | | 50 | μg/ml | 661.7 | −32.7 |

In liver cells (HepG2), recombinant Sclerotium rolfsii lectin of SEQ ID 2 (10 ng/ml-1 μg/ml) demonstrated significant (p<0.001) inhibition of inflammatory cytokine: IL-8 levels as compared to levels induced by Spike S1 protein (SARS-COV-2).

TABLE 16
Inhibitory effect of Recombinant Sclerotium rolfsii lectin
of SEQ ID NO: 2 on expression of inflammatory markers in pancreatic
cells (PANC-1) induced by SpikeS1 protein (SARS-COV-2)

| | | | | Conc. of | |
|---|---|---|---|---|---|
| | | | | IL-8 | % Inhibition |
| SAMPLE | | | | (pg/ml) | (wrt Control ) |
| Untreated | | | | 144.1 | |
| Spike S1 Protein (5 μg/ml) | | | | 746.3 | 0.0 |
| Spike S1 | Dexamethasone | 0.1 | μM | 330.5 | 55.7 |
| Protein | (PC-1) | 1 | μM | 296.2 | 60.3 |
| (20 μg/ml)+ | | 10 | μM | 342.8 | 54.1 |
| | | 20 | μM | 255.7 | 65.7 |
| | Hydroxy- | 0.1 | μM | 214 | 71.3 |
| | chloroquine | 1 | μM | 169.9 | 77.2 |
| | sulphate | 10 | μM | 200.5 | 73.1 |
| | (PC-2) | 20 | μM | 203 | 72.8 |
| | SEQ ID : 2 | 10 | ng/ml | 142.9 | 80.9 |
| | | 100 | ng/ml | 126.9 | 83.0 |
| | | 1 | μg/ml | 168.6 | 77.4 |
| | | 10 | μg/ml | 478.9 | 35.8 |

In pancreatic cells (PANC-1), recombinant Sclerotium rolfsii lectin of SEQ ID 2 (10 ng/ml-10 μg/ml) demonstrated significant (p<0.001) inhibition of inflammatory cytokine; IL-8 levels as compared to levels induced by Spike S1 protein (SARS-COV-2).

We claim:

1. A recombinant Sclerotium rolfsii lectin protein of SEQ ID NO. 1 or an amino acid sequence having at least 70% homology to SEQ ID NO. 1; for use in the treatment or prevention of an infectious disease in a subject caused by Coronaviridae or a mutant thereof.

2. The recombinant Sclerotium rolfsii lectin protein of claim 1, wherein the amino acid sequence has at least 75%, 80%, 90%, 95%, 96%, 97%, 98% or 99% homology to SEQ ID NO. 1.

3. The recombinant Sclerotium rolfsii lectin protein of claim 1, wherein the lectin protein is selected from the group consisting of SEQ ID NO. 2, SEQ ID NO. 3 and SEQ ID NO. 4.

4. The recombinant Sclerotium rolfsii lectin protein of claim 1, wherein the recombinant lectin protein inhibits the viral entry in a cell by blocking SpikeS1 protein of SARS-COV2 from binding with ACE2 receptors.

5. The recombinant Sclerotium rolfsii lectin of claim 1, wherein the recombinant lectin protein modulates the cytokine storm induced by SpikeS1 proteins of coronaviridae or a mutant thereof by inhibition of cytokines such as IL6 and IL8.

6. The recombinant Sclerotium rolfsii lectin protein of claim 1, wherein the recombinant lectin protein interrupts SARS-COV-2 entry in a cell by:
a) AT1R downregulation, or
b) Inhibition of proteases such as Cathepsin L, 3 CL, DPP4.

7. Use of recombinant Sclerotium rolfsii lectin protein of claim 1 for the manufacture of a medicament for use in the treatment or prevention of an infectious disease caused by Coronaviridae or a mutant thereof.

8. A pharmaceutical composition comprising a recombinant Sclerotium rolfsii lectin protein of SEQ ID NO: 1 or an amino acid sequence having at least 70% homology to SEQ ID NO. 1; and a pharmaceutically acceptable diluent or excipient for use in the treatment or prevention of an infectious disease caused by Coronaviridae or a mutant thereof.

9. The pharmaceutical composition of claim 8, wherein the infectious disease caused by Coronaviridae or a mutant thereof is a disease caused by SARS-COV-2 SARS-COV, MERS-CoV, HCoV-229E, HCoV-OC43, HCoV-NL63, HCoV-HKU1, and mutants thereof.

10. Use of a composition of claim 8 for the manufacture of a medicament for use in the treatment or prevention of an infectious disease caused by Coronaviridae or a mutant thereof.

11. A method of treating or preventing an infectious disease caused by Coronaviridae or a mutant thereof in a subject, the method comprising administering to the subject a therapeutically effective amount of a recombinant Sclerotium rolfsii lectin protein of SEQ ID NO: 1 or an amino acid sequence having at least 70% homology to SEQ ID NO. 1.

12. The method of claim 11, wherein the infectious disease caused by Coronaviridae or a mutant thereof is a disease caused by one or more viruses selected from SARS-COV-2, SARS-COV, MERS-COV, HCOV-229E, HCoV-OC43, HCoV-NL63, HCoV-HKU1, and mutants thereof.

13. The method of claim 11, wherein the recombinant lectin protein inhibits the interaction or binding of SpikeS1 protein of SARS-COV2 with ACE2 receptor; and inhibits or modulates the cytokine storm induced by SpikeS1 proteins of coronaviridae or a mutant thereof.

14. The method of claim 11, wherein said treatment or prevention comprises administering the lectin protein at a dose of 1 to 50 mg/kg.


SEQUENCE LISTING (4 sequences)

SEQ ID NO: 1 - protein, 141 aa, synthetic construct
TYKITVRVYQTNPNAFFHPVEKTVWKYANGGTWTITDDQHVLTMGGSGTSGTLRFHADNGESFTATFGVHNYKRWCDIVTNLAADETGMVINQQYYSQKNREEARERQLSNYEVKNAKGRNFEIVYTEAEGNDLHANLIIG

SEQ ID NO: 2 - protein, 141 aa, synthetic construct
TYKITVRVYQTNPDAFFHPVEKTVWKYANGGTWTITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 3 - protein, 141 aa, synthetic construct
VYKITVRVYQTNPDAFFHPVEKTVWKYANGGTWSITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 4 - protein, 141 aa, synthetic construct
VYKITVRVYQTNPDAFFHPVEKTVWKYADGGTWSITDDQHVLTMGGSGTSGTLRFHADNGESFTATFGVHDYKRWCDIVTDLAADETGMVINQEYYSEKDREEARERQNSNYEVKDAKGRNFEIVYTEAEGNDLHADLIIG